(19) United States
(12) Patent Application Publication
(10) Pub. No.: US 2007/0110711 A1
(43) Pub. Date: May 17, 2007

(54) Agent for enhancing the production of cytokines and/or chemokines

(75) Inventors: Makoto Takeuchi, Okayama (JP); Takanori Okura, Okayama (JP); Tomoki Tatefuji, Okayama (JP); Tetsuya Mori, Okayama (JP); Chikako Arai, Okayama (JP); Tsunetaka Ohta, Okayama (JP); Masashi Kurimoto, Okayama (JP)

Correspondence Address: BROWDY AND NEIMARK, P.L.L.C.;624 NINTH STREET, NW, SUITE 300, WASHINGTON, DC, 20001-5303, US

(73) Assignee: KABUSHIKI KAISHA HAYASHIBARA SEIBUTSU KAGAKU KENKYUJO, Okayama (JP)

(21) Appl. No.: 11/583,148

(22) Filed: Oct. 19, 2006

(30) Foreign Application Priority Data
Oct. 14, 2005 (JP) 2005-299571
Oct. 14, 2004 (JP) 2004-300786

(51) Int. Cl. A61K 38/19; C07K 14/52

(52) U.S. Cl. 424/85.1; 530/351

(57) ABSTRACT

The present invention has an object to provide a means to effectively enhance the production of cytokines and/or chemokines in mammals. The object is solved by providing an agent for enhancing the production of cytokines and/or chemokines, which comprises, as an effective ingredient, a polypeptide having any one of the amino acid sequences of SEQ ID NOs:1 to 3; a polypeptide having any one of the amino acid sequences of SEQ ID NOs:1 to 3, where one or more amino acids thereof are deleted or replaced with other amino acid(s) and/or one or more amino acids are added thereunto, without substantially losing the biological activity of the polypeptide.

### TECHNICAL FIELD

The present invention relates to an agent for enhancing the production of cytokines and/or chemokines, more particularly, to an agent for enhancing the production of cytokines and/or chemokines, which contains as an effective ingredient either a polypeptide having any one of the amino acid sequences of SEQ ID NOs:1 to 3; or a polypeptide having any one of the amino acid sequences of SEQ ID NOs:1 to 3, where one or more amino acids thereof are deleted or replaced with other amino acid(s) and/or one or more amino acids are added thereunto, without substantially losing the biological activity of the polypeptide.

### BACKGROUND ART

Living bodies exhibit biological defence reactions against external physical-, chemical-, or biological-invaders, in which various cytokines and chemokines are involved. However, in the case that living bodies are out of their normal function due to any causatives, for example, aging, cancer, chemotherapy after cancer treatment, or decline in physical strength due to infection, there have been made many trials to assist biological reactions by externally supplementing such cytokines or chemokines.

Thrombopoietin (TPO) has been expected for use as a method for treating recently-notable problematic thrombocytopenia induced by chemotherapy after cancer treatment, however, the effect is not clear as disclosed in, for example, “Igaku-no-Ayumi”, Ishiyaku Publisher Inc., Tokyo, Japan, Vol. 190, No. 10, pp. 890-895 (1999). Interleukin-6 (IL-6) has been known to be produced during inflammation and have functions to increase platelet blood count, as well as to differentiate and maturate megakaryocyte in bone marrow, and therefore it has been said that IL-6 is clinically useful in treating patients suffering from thrombocytopenia. Recently, macrophage colony-stimulating factor (M-CSF), originally found as a factor of stimulating the formation of monocytes and macrophages, has been also known to have a function of increasing platelet blood count and has been expected for use as an agent for increasing platelet blood count to be used after chemotherapy. It has been also known that interleukin-1 (IL-1) has an action of activating hematopoiesis, as disclosed in, for example, “Cytokine therapy—Approach from basic and pathological states”, pp. 74-79 (1992), Nankodo Co., Ltd., Tokyo, Japan; and “All About Cytokines”, Vol. 27, No. 16, pp. 551-561 (1995), Kagaku-Hyoronsha Co., Ltd., Tokyo, Japan. Accordingly, if there exists any method for efficiently producing autogenous IL-6, M-CSF and/or IL-1, that act on not only platelet but other hematopoietic system, it could possibly increase platelet blood count in patients while decreasing side effects compared with that administered with IL-6 or other factors, produced on an industrial scale by means of recombinant technology, etc.

While, cytokines such as transforming growth factor-β (TGF-β), tumor necrosis factor-α (TNF-α), and vascular endothelial growth factor (VEGF) are known to differentiate and proliferate cells, repair tissue, and induce angiogenesis, as well as to promote the production of collagen and fibronectin, and thus they have been expected to be applied for recovering or treating wounded tissues, as disclosed in, for example, “All About Cytokines”, Vol. 27, No. 16, pp. 551-561 (1995), Kagaku-Hyoronsha Co., Ltd., Tokyo, Japan; and “Jikken-Igaku”, Vol. 17, No. 6, pp. 721-726 (1999), Yodosha Co., Ltd., Tokyo, Japan.

Further, in these recent studies, it was unexpectedly found that chemokines, once found as factors that induce hemocytes in local inflammatory sites, also have a novel biological activity in addition to their stimulation of cell migration. For example, the following have been found: Stromal cell derived factor-1 (SDF-1) has an activity of functioning as a multi-functional cytokine that relates to organogenesis such as hematopoiesis, heart formation, brain formation, angiogenesis of stomach and enteron during fetal life mainly; interleukin-8 (IL-8) has an activity of inhibiting cytomegalovirus in fibroblasts; and both of regulated upon activation, normal T-cell expressed and secreted (RANTES) and macrophage inflammatory protein (MIP) have an activity of inhibiting the infection of human immunodeficiency virus-(HIV). It was also revealed that Duffy antigen, as an antigen of infected Plasmodium vivax, binds to most of chemokines, and HIV infects macrophage and T-cells by recognizing their chemokine receptors, for example, SDF-1, RANTES and MIP. Thus, there has been being revealed that chemokines per se possibly inhibit the infection of pathogens on a receptor level, as disclosed in, for example, “Kensho-Chemokines”, Vol. 17, No. 7, pp. 1082-1089 (1998), Shujunsha Co., Ltd., Tokyo, Japan. Accordingly, it would be expected that chemokines would have an effect such as the prevention of the infection of HIV and other pathogens when they are effectively produced in vivo or in vitro.

Under these circumstances, it has been desired a means for effectively enhance the in vivo or in vitro production of cytokines and chemokines because the finding thereof would possibly become the one useful as an inducer in producing cytokines and chemokines, a strong auxiliary means for ameliorating thrombocytopenia induced during cancer chemotherapy or promoting the recovery of health from such disorder, and further a protection means for preventing living bodies from pathogenic infection by using biological functions.

### DISCLOSURE OF INVENTION

In view of the above background, an object of the present invention is to provide a means for effectively enhancing the production of cytokines and chemokines in mammals.

The present inventors focused on both a human polypeptide, AgK114-1a, as disclosed in International Publication No. WO 2004/042056 applied for by the same applicant as the present invention, i.e., a polypeptide having the amino acid sequence of SEQ ID NO:1; and a murine polypeptide, mAgK114-1b and mAgK114-1, i.e., polypeptides having the amino acid sequences of SEQ ID NOs:2 and 3, respectively, and they found that, when allowed to act on mammalian mesenchyme cells, epithelial cells, or macrophage cells in vitro, the above-identified polypeptides remarkably enhance the production of IL-6 in the above-identified cells, as well as the production of M-CSF and IL-1 as cytokines capable of recovering the platelet blood count similarly as in IL-6, other cytokines such as TNF-α, TGF-β and VEGF, and chemokines such as SDF-1, RANTES, IL-8 and MIP.

The present invention solves the above object by providing an agent for enhancing the production of cytokines and/or chemokines, which contains as an effective ingredient a polypeptide either having any one of the amino acid sequences of SEQ ID NOs:1 to 3, or having any one of the amino acid sequences of SEQ ID NOs:1 to 3, where one or more amino acids thereof are deleted or replaced with other amino acid(s) and/or one or more amino acids are added thereunto, without substantially losing the biological activity of the polypeptide.

Since the agent for enhancing the production of cytokines and/or chemokines according to the present invention remarkably enhances the production of IL-6, M-CSF and IL-1 in mammalian cells, it enhances the hematopoietic action of the above cytokines and/or chemokines. Thus, the agent of the present invention is useful in the field of pharmaceuticals for increasing the number of blood cells such as platelet reduced in number by chemotherapy, radiotherapy in cancer treatment, or bone marrow transplantation, and for successively proliferating hematopoietic cells in vitro and then administering the proliferated hematopoietic cells to living bodies. Since the agent of the present invention also augments the production of cytokines such as TNF-α, TGF-β and VEGF, they are useful in repairing wounded tissues. The agent for enhancing the production of cytokines and/or chemokines according to the present invention further augments the production of chemokines such as SDF-1, RANTES, IL-8 and MIP, and it is useful as an agent for alleviating symptom such as atopic dermatitis and for preventing the infection of microorganisms, etc.

### BRIEF DESCRIPTION OF DRAWINGS

FIG. 1 is a figure of intermediate image for a digital image of microscopic photograph of murine skin slice as a control, displayed on a display.

FIG. 2 is a figure of intermediate image for a digital image of microscopic photograph of murine skin slice applied with mAgK114-1b, displayed on a display.

### BEST MODE FOR CARRYING OUT OF THE INVENTION

The agent for enhancing the production of cytokines and/or chemokines according to the present invention is an agent which contains, as an effective ingredient, either a polypeptide having any one of the amino acid sequences of SEQ ID NOs:1 to 3, disclosed in International Publication No. WO 2004/042056, applied for by the same applicant as the present invention; or a polypeptide having any one of the amino acid sequences of SEQ ID NOs:1 to 3, where one or more amino acids thereof are deleted or replaced with other amino acid(s) and/or one or more amino acids are added thereunto, without substantially losing the biological activity of the polypeptide. These polypeptides should not be restricted to ones with a specific purity, origin or preparation method as long as they have any one of the above amino acid sequences and exert an action of enhancing the production of cytokines and/or chemokines in vivo or in vitro. The term “a polypeptide having any one of the amino acid sequences of SEQ ID NOs:1 to 3, where one or more amino acids thereof are deleted or replaced with other amino acid(s) and/or one or more amino acids are added thereunto, without substantially losing the biological activity of the polypeptide” as referred to as in the present invention means a polypeptide which has any one of the amino acid sequences of SEQ ID NOs:1 to 3, where an amino acid residue(s) of any one of the amino acid sequences of SEQ ID NOs:1 to 3 is(are) replaced with other amino acid residue(s), 1 to 10 amino acid residues in any one of the amino acid sequences of SEQ ID NOs:1 to 3 are deleted, or 1 to 60 amino acids are added to or inserted into the N- and/or C-termini or the internal site(s) of the N- and/or C-terminal regions of any one of the amino acid sequences of SEQ ID NOs:1 to 3. For example, polypeptides having any one of the amino acid sequences of SEQ ID NOs:1 to 3, where one or more alanine residues therein are replaced with glycine residues, one or more alanine residues therein are deleted, or one or more alanine residues are newly added thereunto. These polypeptides with mutated amino acid sequences can be obtained by using protein engineering technique such as site-specific mutagenesis and random mutagenesis. The presence or the absence of the action of enhancing the production of cytokines and/or chemokines in mammals can be determined by means of culturing a neonatal normal human dermal fibroblast cell line (NHDF cell), mouse embryonic fibroblast cell (mesenchyme cell) line (MEF cell), or mouse macrophage cell line (J774A.1 cell) in the presence or the absence of the testing polypeptide, and assaying the production level of cytokines and/or chemokines in each culture supernatant.

The polypeptides used in the present invention include any polypeptides as long as they have any one of the above-identified amino acid sequences and enhance the production of cytokines and/or chemokines in mammals. Examples of such polypeptides are those which are prepared by recombinant DNA technology, those which are derived from natural sources, and those which are chemically synthesized. Further, artificially, chemically modified ones, which are prepared by binding water-soluble natural or synthetic high molecules such dextran, pullulan or polyethylene glycol (PEG) with an average molecular weight of 5,000 to 10,000, can be arbitrarily used.

These polypeptides having those amino acid sequences can be produced by preparing transformed cells or microorganisms capable of producing any one of such polypeptides by recombinant DNA technology using a DNA which encodes any one of the polypeptides, and culturing the transformed cells or microorganisms to produce the desired polypeptides intracellularly or extracellularly. The term “DNA” as referred to as in the present invention means one which encodes any one of the polypeptides used in the present invention. Examples of such are the nucleotide sequences of SEQ ID NOs:4 to 6 which encode the amino acid sequences of SEQ ID NOs:1 to 3, those which one or more bases in any one of the nucleotide sequences of SEQ ID NOs:4 to 6 are replaced with other bases without substantially altering the amino acid sequence encoded by any one of the nucleotide sequences of SEQ ID NOs:4 to 6, and those which are complementary ones to the above nucleotide sequences. These DNAs should not specifically be restricted to those of natural origins or artificially synthesized ones. Examples of the sources of DNAs used in the present invention are illustrated with human placental cells and mouse skin cells, which can be prepared by the method as disclosed in International Publication No. WO 2004/042056, applied for by the same applicant as the present invention.

The polypeptides used in the present invention can be also prepared by chemical synthesis based on the amino acid sequences of SEQ ID NOs:1 to 3; the peptide synthetic methods used in the present invention include any of those which employ peptide synthesizers generally used in this field to form the desired whole peptides, and those which previously synthesize peptide fragments in separate blocks and then condensing them enzymatically or chemically. These methods can be arbitrarily used, depending on use.

Crude preparations of the polypeptides usable in the present invention, which are obtainable by using recombinant DNA technology or peptide synthesis, or obtainable from natural sources, can be used intact as production enhancers for cytokines and/or chemokines in mammals, however, they may be usually purified prior to use. The methods for purifying the polypeptides used in the present invention include those which are used in general in this art to purify biologically active polypeptides; concentration, salting out, dialysis, separatory sedimentation, gel filtration chromatography, ion-exchange chromatography, hydrophobic chromatography, affinity chromatography, chromatofocusing, gel electrophoresis, and isoelectric focusing, which can be used in an appropriate combination, if necessary. Depending on final use, the purified polypeptides thus obtained can be concentrated and/or lyophilized into a liquid or solid preparation.

The term “cytokines” as referred to as in the present invention means one or more cytokines selected from interleukin-6 (IL-6), macrophage colony-stimulating factor (M-CSF), interleukin-1 (IL-1), tumor necrosis factor-α (TNF-α), transforming growth factor-β (TGF-β), and vascular endothelial growth factor (VEGF); and the term “chemokines” as referred to as in the present invention means one or more chemokines selected from stromal cell derived factor-1 (SDF-1); regulated on activation, normal T-cell expressed and secreted (RANTES); interleukin-8 (IL-8); and macrophage inflammatory protein (MIP). As described above, since the polypeptides used in the present invention have an action of enhancing the production of the above-identified cytokines and/or chemokines, they can be advantageously used for uses in the field of pharmaceuticals, etc., that require substances with such an action. In the field of pharmaceuticals, the polypeptides are useful as factors for increasing platelet blood count. The polypeptides can be advantageously used to produce chemokines and used as agents for alleviating inflammation, as well as agents in the form of an external skin agent to alleviate atopic dermatitis, contact hypersensitivity, and their accompanying skin inflammations. The polypeptides used in the present invention are substances with quite low toxicity and satisfactory safeness because they are intrinsically mammalian origin.

The higher the content of the polypeptide(s) as an effective ingredient(s) contained in the agent for enhancing the production of cytokines and/or chemokines of the present invention, the more remarkably the agent exhibits an action of enhancing the production of cytokines and/or chemokines. In the present invention, the polypeptides in a highly or partially purified form can be used, however, as stated in the following Examples where the tests for enhancing the production of cytokines and/or chemokines are conducted using polypeptide preparations with a concentration of 10 μg/ml of the polypeptide(s), it is preferable to increase the polypeptide content in the agent to a level that makes the agent increase the relative production levels of cytokines and/or chemokines by at least two fold higher than that attained without using any polypeptides.

Explaining the use of the agent for enhancing the production of cytokines and/or chemokines according to the present invention, the agent can be used as an inducer to be supplemented to culture media in producing cytokines and/or chemokines by means of cell culture. Cells such as mesenchyme cells, fibroblasts, epithelial cells, and hematopoietic cells which are separated from mammalian skin, oral cavity, bone marrow, and blood; and established cell lines such as MEF cells and J774A.1 cells are cultured in appropriate culture media containing about 0.1 ng/ml to about 100 μg/ml, preferably, about 1 to about 50 μg/ml of the polypeptide(s) of the present invention. If necessary, cell-stimulating substances such as mitogens can be added to the culture media for these cells, followed by culturing the above cells for about 1 to about 100 hours in conventional manner while keeping at a temperature of about 30 to about 40° C. and a pH of about 5 to about 8 and appropriately replacing the media with fresh ones. The desired cytokines and/or chemokines can be collected by applying to the resulting cultures the following one or more techniques in an appropriate combination; salting out, dialysis, filtration, concentration, separatory sedimentation, gel filtration chromatography, ion-exchange chromatography, hydrophobic chromatography, affinity chromatography, chromatofocusing, gel electrophoresis, and isoelectric focusing.

By administering to living bodies, the agent for enhancing the production of cytokines and/or chemokines according to the present invention can be used as a therapeutic agent for thrombocytopenia and tissue disorder in living bodies and used as a preventive to protect the infection of external microorganisms. To treat and/or prevent thrombocytopenia, tissue disorder, and infectious diseases, the agent for enhancing the production of cytokines and/or chemokines can be directly applied or administered to mammalian bodies. The effective dose of the polypeptides, as effective ingredients, of the agent may vary depending on the kind, age, sexuality, etc., of mammals including humans to be administered therewith, the agent is prepared into an appropriate form suitable for administration to the mammals orally or transmucosally, such as intracutaneous, subcutaneous, intramuscular, intravenous, and intraperitoneal injections. The agent for the production enhancer for cytokines and/or chemokines according to the present invention is usually administered at a dose of 1 to 1,000 μg/shot, desirably, 10 to 500 μg/shot in terms of the amount of the polypeptide(s) as effective ingredient(s) of the agent at a frequency of one to several shots per day and at successive days or at intervals of one or more days, depending on the symptom and the administration form or route. The mammals, administrable with the agent for enhancing the production of cytokines and/or chemokines according to the present invention, should not be restricted to humans and include mice, rats, hamsters, rabbits, dogs, cats, cows, horses, goats, sheep, pigs, apes/monkeys, etc. Since the polypeptides used as effective ingredients in the present invention are quite low in toxicity, they do not substantially cause serious side effects even when administered at a relatively high dose. Thus, the agent for enhancing the production of cytokines and/or chemokines according to the present invention has the merit that it quickly induces the desired cytokines and/or chemokines without strict dose control when in use.

The compositions incorporated with the agent for enhancing the production of cytokines and/or chemokines according to the present invention can be arbitrarily used in the form of a pharmaceutical. The agent can be arbitrarily incorporated with one or more pharmaceutically acceptable other ingredients for mammals including humans, such as water, alcohols, amylaceous substances, proteins, amino acids, fibers, saccharides, lipids, fatty acids, vitamins, minerals, flavors, colors, sweeteners, seasonings, spices, stabilizers, antiseptics, emulsifiers, surfactants, excipients, fillers, thickeners, preservatives, etc. These ingredients are appropriately selected depending on the necessity for the fields applied with the agent for enhancing the production of cytokines and/or chemokines according to the present invention. The agent containing the above ingredients should not be restricted to the one in a specific form and it can be provided in a desired form such as a powder, granule, tablet, paste, jelly, emulsion, or liquid.

The saccharides as mentioned above include, for example, saccharides such as glucose, fructose, lactose, trehalose, maltose, sucrose, lactosucrose, and starch syrup; cyclic saccharides such as cyclodextrins and cyclic tetrasaccharides; sugar alcohols such as erythritol., mannitol, sorbitol, xylitol, maltitol, and hydrogenated starch syrups; natural polysaccharides such as pullulan and carrageenan; natural gums; and carboxymethyl cellulose, one or more of which can be added to the agent for enhancing the production of cytokines and/or chemokines according to the present invention. Among the above saccharides, those in a solid form can be arbitrarily used as an excipient and a stabilizer for the agent for enhancing the production of cytokines and/or chemokines according to the present invention.

The compositions incorporated with the agent for enhancing the production of cytokines and/or chemokines according to the present invention are prepared by mixing the agent with one or more of the above exemplified ingredients accepted for use in the field of pharmaceuticals based on their respective contents and final use and according to their appropriate compositions selected depending on animals/mammals to be administered therewith and their administration routes; appropriately employing the steps of dilution, concentration, drying, filtration, centrifugation, etc.; and optionally forming the resulting mixtures into products in a desired form. The order of incorporating the ingredients and the timing of applying the above steps are not specifically restricted as long as they do not deteriorate the quality of the objective agent for enhancing the production of cytokines and/or chemokines of the present invention, and any of the above steps can be appropriately employed depending on use.

Examples of the preferred pharmaceutical forms of the agent are extracts, elixirs, capsules, granules, pearls/pills, ophthalmic ointments, adhesive preparations for oral mucous membrane, suspensions, emulsions, plasters, suppositories, powdered medicines, spirits, syrups, injections, tinctures, eye drops, ear drops, collunariums, trochees, ointments, waters, nasal nebulae, limonades, liniments, fluidextracts, lotions, medicines for stupe, nebulae, embrocations, bath preparations, adhesive preparations, pastes, and cataplasms. The compositions, incorporated with the agent for enhancing the production of cytokines and/or chemokines according to the present invention, can be prepared by adding the agent to the materials of the desired compositions at an appropriate timing during their processings according to conventional production methods. The timing of the above addition should not specifically be restricted, however, in the case of the objective products are prepared through a heating step, the agent should preferably be added after a cooling step at ambient temperature, preferably, at a temperature of 30° C. or lower to prevent the reduction of the activity of enhancing the production of cytokines and/or chemokines in the agent during their production steps. The compositions of the present invention thus obtained contain the agent for enhancing the production of cytokines and/or chemokines in an amount of, usually, at least 0.01% by weight, preferably, 0.1 to 100% by weight to each composition by weight.

As described above, since the agent for enhancing the production of cytokines and/or chemokines according to the present invention enhances the production of cytokines and/or chemokines in hematopoietic cells including mammalian mesenchyme cells, fibroblasts, epithelial cells, and macrophage cells, it effectively exerts the action of enhancing the production of cytokines and/or chemokines in living bodies administered therewith without causing serious side effects, followed by exerting effect on the increment of platelet blood count, phylactic effect on phagocytes, inhibitory effect on allergy, etc.

The following Examples explain the present invention in more detail but they do not limit the scope of the present invention.

### EXAMPLE 1

### Action of Enhancing the Production of Interleukin-6 (IL-6) in Mouse Embryonic Fibroblasts (MEF cells) and Mouse Macrophage Cells by Mouse Polypeptide mAqK114-1b

Mouse polypeptide mAgK114-1b, i.e., a polypeptide having the amino acid sequence of SEQ ID NO:2, which had been prepared and purified by the method in Example 10 disclosed in International Publication No. WO 2004/042056, was studied for its action of enhancing the production of IL-6 in mouse embryonic fibroblast cell line (MEF cells) and mouse macrophage cell line (J774A.1 cells). According to conventional manner, MEF cells or J774A.1 cells were inoculated to a 24-well microplate with two milliliters of D-MEM medium supplemented with 10% (v/v) fetal calf serum, at a cell density of about 2×10⁴ cells/ml. The cells were incubated overnight at 37° C. under 5% (v/v) CO₂ conditions. To the proliferated MEF cells or J774A.1 cells was added two milliliters of a serum-free Dulbecco's Modified Eagle Medium (D-MEM, commercialized by Nissui Pharmaceutical Co. Ltd., Tokyo, Japan) supplemented with a purified. mAgK114-1b preparation to give a final concentration of 0.1 μg/ml, 1 μg/ml or 10 μg/ml, followed by further incubation at 37° C. for two days under 5% (v/v) CO₂ conditions. After completion of the culture, culture supernatants were collected from the resulting cultures by centrifugation and assayed for IL-6 level in each culture supernatant by using enzyme-linked immunosorbent assay (ELISA). As a control, a culture supernatant prepared from a cell culture with no addition of the purified mAgK114-1b preparation. The result is in Table 1.

TABLE 1

| | |
|---|---|
| | Production level |
| Concentration of | (ng/ml) of IL-6* |

| polypeptide (μg/ml) | MEF cells | J774A.1 cells |
|---|---|---|
| 0.0 | ND** | 4.74 |
| 0.1 | ND | 5.33 |
| 1.0 | ND | 6.31 |
| 10.0 | 0.72 | 22.81 |

*Interleukin-6
**Below detectable limit of 0.028 ng/ml

As evident from the result in Table 1, when used at concentrations of 1 μg/ml and 10 μg/ml, the mouse polypeptide mAgK114-1b dose-dependently enhanced the production level of IL-6 in J774A.1 cells by about 1.3-folds and about 4.8-folds higher than that of the control free of the polypeptide, respectively. While, the production level of IL-6 in MEF cells cultured in the serum-free system was below its detectable limit but the level was increased to 0.72 ng/ml when the cells were cultured at a concentration of 10 μg/ml of the polypeptide. This experiment revealed that the mouse polypeptide mAgK114-1b remarkably enhances the production of IL-6 in MEF cells or J774A.1 cells in a dose dependent manner. Thus, the polypeptide used in the present invention remarkably enhances the production of IL-6 in mesenchyme cells and macrophage cells and it can be used as a factor for increasing platelet blood count.

### EXAMPLE 2

### Action of Enhancing the Production of Interleukin-6 (IL-6) in Neonatal Normal Human Dermal Fibroblasts by Human Polypeptide hAgK114-1a FL

Human polypeptide hAgK114-1aFL, i.e., a polypeptide having the amino acid sequence of SEQ ID NO:7, i.e., a sequence of FLAG which positions at the C-terminus of the polypeptide having the amino acid sequence of SEQ ID NO:1, which had been prepared and purified by the method in Example 7 disclosed in International Publication No. WO 2004/042056, was studied for its action of enhancing the production of IL-6 in neonatal normal human dermal fibroblast cell line (NHDF cells). According to conventional manner, NHDF cells were inoculated to a 6-well microplate with two milliliters of D-MEM medium supplemented with 10% (v/v) fetal calf serum, at a cell density of about 5×10⁵ cells/ml. The cells were incubated overnight at 37° C. under 5% (v/v) CO₂ conditions. The culture medium of the proliferated NHDF cells was replaced with a serum-free D-MEM supplemented with a purified hAgK114-1aFL preparation to give a final concentration of 1 μg/ml or 10 μg/ml, followed by further incubation at 37° C. for two days under 5% (v/v) CO₂ conditions. After completion of the culture, culture supernatants were collected from the resulting cultures by centrifugation and assayed for IL-6 protein level in each culture supernatant by using ELISA. As a control, a culture supernatant prepared from a cell culture without using the purified hAgK114-1aFL preparation. The result is in Table 2.

TABLE 2

| Concentration of | Production level of IL-6* |
|---|---|
| polypeptide (μg/ml) | (pg/ml) |
| 0.0 | 1.76 |
| 1.0 | 7.04 |
| 10.0 | 3.28 |

*Interleukin 6

As evident from the result in Table 6, when used at concentrations of 1 μg/ml and 10 μg/ml, the human polypeptide hAgK114-1aFL enhanced the production level of IL-6 in NHDF cells by about 4-folds and about 1.9-folds higher than that of the control free of the polypeptide, respectively. This experiment revealed that the human polypeptide hAgK114-1aFL remarkably enhances the production of IL-6 in NHDF cells. Thus, the polypeptide used in the present invention remarkably enhances the production of IL-6 in mesenchyme cells and it can be used as a factor for increasing platelet blood count.

### EXAMPLE 3

### Action of Enhancing the Production of Macrophage Colony-Stimulating Factor (M-CSF) in Mouse Embryonic Fibroblasts (MEF Cells) and Mouse Macrophage Cells by Mouse Polypeptide mAqK114-1b

Using culture supernatants prepared from cultures of MEF cells and J774A.1 cells obtained by the method in Example 1, the production level of M-CSF protein in each culture supernatant was assayed on an enzyme immunoassay (EIA) kit commercialized by R & D System Inc., Minneapolis, Minn., USA.

The result is in Table 3.

TABLE 3

| | |
|---|---|
| | Production level |
| Concentration of | (ng/ml) of M-CSF* |

| polypeptide (μg/ml) | MEF cells | J774A.1 cells |
|---|---|---|
| 0.0 | 0.09 | 0.95 |
| 0.1 | 0.09 | 1.25 |
| 1.0 | 0.10 | 1.50 |
| 10.0 | 0.12 | 3.53 |

*Macrophage colony-stimulating factor

As evident from the result in Table 3, when used at concentrations of 1 μg/ml and 10 μg/ml, the mouse polypeptide mAgK114-1b dose-dependently enhanced the production level of M-CSF in J774A.1 cells by about 1.6-folds and about 3.7-folds higher than that of the control free of the polypeptide, respectively. While, no difference was found in MEF cells cultured with or without the polypeptide. This experiment revealed that the mouse polypeptide mAgK114-1b remarkably enhances the production of M-CSF in J774A.1 cells in a dose dependent manner. Thus, the polypeptide used in the present invention remarkably enhances the production of M-CSF in macrophage cells and it can be used as a factor for increasing platelet blood count.

### EXAMPLE 4

### Action of Enhancing the Production of Interleukin-1 (IL-1) and Tumor Necrosis Factor-α (TNF-α) in Mouse Embryonic Fibroblasts (MEF Cells) and Mouse Macrophage Cells by Mouse Polypeptide mAqK114-1b

Similarly as in Example 1, except for using a purified mouse polypeptide mAgK114-1b preparation was used at a concentration of 10 μg/ml, it was conducted cell culture and the resulting cell culture supernatants were assayed for the level of interleukin-1α and interleukin-1β proteins and TNF-α protein by using an enzyme immunoassay (EIA) kit commercialized by R & D System, Minneapolis, Minn., USA. As a control, it was used a supernatant prepared from cell cultures without addition of the purified mouse polypeptide mAgK114-1b preparation. The results are in Tables 4 and 5.

TABLE 4

| | | |
|---|---|---|
| Concentration of | | |
| polypeptide (μg/ml) | MEF cells | J774A.1 cells |

Production level
(pg/ml) of IL-1α*

| | | |
|---|---|---|
| 0.0 | ND | ND*** |
| 10.0 | ND | 88.7 |

Production level
(pg/ml) of IL-1β**

| | | |
|---|---|---|
| 0.0 | ND | ND**** |
| 10.0 | ND | 24.8 |

*Interleukin-1α
**Interleukin-1β
***Below detectable limit of 9.4 pg/ml
****Below detectable limit of 15.6 pg/ml

TABLE 5

| | |
|---|---|
| | Production level |
| Concentration of | (pg/ml) of TNF-α* |

| polypeptide (μg/ml) | MEF cells | J774A.1 cells |
|---|---|---|
| 0.0 | ND | ND** |
| 10.0 | ND | 8369 |

*Tumor necrosis factor-α
**Below detectable limit of 17.8 pg/ml

As evident from the result in Table 4, the mouse polypeptide mAgK114-1b enhanced the production levels of IL-1α and IL-1β in J774A.1 cells by about 9-folds and about 1.6-folds higher than that of the control free of the polypeptide, respectively. While, no production enhancement of the above cytokines in MEF cells was found. As evident from the result in Table 5, considering the detectable limit, the mouse polypeptide mAgK114-1b enhanced the production level of TNF-α in J774A.1 cells by at least about 470-folds higher than that of the control free of the polypeptide, While, no production enhancement of the above cytokine in MEF cells was found. The polypeptide used in the present invention enhances the production of cytokines such as IL-1 and TNF-α and it is useful as an agent for increasing platelet blood count or an agent for repairing tissue used in the field of pharmaceuticals.

### EXAMPLE 5

### Action of Enhancing the Production of Chemokines in Embryonic Fibroblasts (MEF Cells) and Macrophage Cells by Mouse Polypeptide mAqK114-1b

Using culture supernatants prepared from cultures of MEF cells and J774A.1 cells obtained by the method in Example 1, the production levels of chemokines in each culture supernatant were assayed on EIA. The assayed chemokines were SDF-1, IL-8, RANTES and MIP. The results are respectively in Tables 6, 7, 8 and 9.

TABLE 6

| | |
|---|---|
| | Production level |
| Concentration of | (ng/ml) of SDF-1* |

| polypeptide (μg/ml) | MEF cells | J774A.1 cells |
|---|---|---|
| 0.0 | 1.06 | ND** |
| 0.1 | 1.12 | ND |
| 1.0 | 1.03 | ND |
| 10.0 | 2.20 | ND |

*Factor derived from stroma cells
**Below detectable limit of 0.16 ng/ml

TABLE 7

| | |
|---|---|
| | Production level |
| Concentration of | (ng/ml) of IL-8* |

| polypeptide (μg/ml) | MEF cells | J774A.1 cells |
|---|---|---|
| 0.0 | 0.10 | ND** |
| 0.1 | 0.16 | ND |
| 1.0 | 3.52 | ND |
| 10.0 | 21.20 | ND |

*Interleukin 8 (mouse homologue)
**Below detectable limit of 0.016 ng/ml

TABLE 8

| | |
|---|---|
| | Production level |
| Concentration of | (ng/ml) of RANTES* |

| polypeptide (μg/ml) | MEF cells | J774A.1 cells |
|---|---|---|
| 0.0 | 0.03 | 0.26 |
| 0.1 | 0.03 | 0.22 |
| 1.0 | 0.34 | 0.29 |
| 10.0 | 4.98 | 8.43 |

*Gene product expressed in normal T-cells

TABLE 9

| | |
|---|---|
| | Production level |
| Concentration of | (ng/ml) of MIP* |

| polypeptide (μg/ml) | MEF cells | J774A.1 cells |
|---|---|---|
| 0.0 | ND** | 18.2 |
| 0.1 | ND | 22.3 |
| 1.0 | ND | 45.6 |
| 10.0 | 0.02 | 326.0 |

*Macrophage inflammatory protein
**Below detectable limit of 0.0084 ng/ml

As evident from the result in Tables 6 and 7, when used at a concentration of 10 μg/ml, the mouse polypeptide mAgK114-1b enhanced the production levels of SDF-1 and IL-8 in MEF cells by about 2-folds and about 210-folds higher than those of their respective controls free of the polypeptide, respectively. While, no enhancement of the production level of the above chemokines was found in J774A.1 cells. As evident from the result in Table 8, when used at a concentration of 10 μg/ml, the mouse polypeptide mAgK114-1b enhanced the production levels of RANTES in MEF cells and J774A.1 cells by at least about 160-folds and about 32-folds higher than those of their respective controls free of the polypeptide, respectively. Further, as evident from the result in Table 9, the production level of MIP in MEF cells in a system free of the mouse polypeptide mAgK114-1b, as a control, was below the detectable limit, while the polypeptide increased the production level of MIP in MEF cells to 0.02 ng/ml when used at a concentration of 10 μg/ml and increased the production level of MIP in J774A.1 cells by about 18-folds higher than that of the control free of the polypeptide when used at a concentration of 10 μg/ml. The polypeptide used in the present invention enhances the production of chemokines such as SDF-1, interleukin-8 (IL-8), RANTES, and MIP and it is useful as a phylactic agent for pathogens.

### EXAMPLE 6

### Action of Enhancing the Production of Interleukin-6 (IL-6) in Mouse Embryonic Fibroblasts (MEF Cells) and Mouse Macrophage Cells by Mouse Membrane-Associated Polypeptide mAgK114-1

The polypeptide having the amino acid sequence of SEQ ID NO:3, i.e., a mouse membrane-associated polypeptide mAgK114-1, disclosed in International Publication No. WO 2004/042056, was allowed to express on the surface of COS-1 cells and then co-cultured with mouse embryonic fibroblasts (MEF cells) and mouse macrophage cells to examine whether the mouse membrane-associated polypeptide mAgK114-1 enhances the production of IL-6. For comparison, mouse secretory polypeptide mAgK114-1b was examined for its enhancement of the production of IL-6 similarly as above.

### EXAMPLE 6-1

### Construction of Expression Vector of Mouse Membrane-Associated Polypeptide mAgK114-1 and Mouse Secretory Polypeptide mAgK114-1b

By using as templates the recombinant plasmids pTB-mAgK114PCR13 and pTB-mAgK114PCR181, which are respectively disclosed in Examples 3 and 4 in International Publication No. WO 2004/042056, DNAs encoding membrane-associated polypeptide mAgK114-1 and secretory polypeptide mAgK114-1b were prepared for use to construct expression vectors. By using 10 nano grams aliquots of each of the plasmids as templates, PCR was conducted to obtain pTB-mAgK114PCR13 using both a synthetic DNA having the nucleotide sequence of SEQ ID NO:8 as a sense primer, and a synthetic DNA having the nucleotide sequence of SEQ ID NO:9 as a complementary chain primer. Further, PCR was conducted to obtain pTB-mAgK1l4PCR181 by using both a synthetic DNA having the nucleotide sequence of SEQ ID NO:10 as a sense primer, and a synthetic DNA having the nucleotide sequence of SEQ ID NO:11 as a complementary chain primer. These two amplified fragments thus obtained were purified by sedimentation with polyethylene glycol and cloned to the site of Srf I of plasmid vector pCR-Script CamSK(+) commercialized by Stratagene Japan K.K., Tokyo, Japan. According to a conventional manner, these amplified fragments were confirmed their nucleotide sequences, revealing that the DNA, which had been constructed to encode the desired membrane-associated polypeptide mAgK114-1, had the nucleotide sequence of SEQ ID NO:6; while the DNA, which had been constructed to encode the desired secretory polypeptide mAgK114-1b, had a nucleotide sequence of SEQ ID NO:5, where a nucleotide sequence that recognizes Xho I was added to the 5′-terminus and the one that recognizes Not I was added to the 3′-terminus. A Xho I-Not I fragment containing the resulting cDNA was cut out again and inserted therein an expression vector pCDM8 commercialized by Invitrogen Japan K.K., Tokyo, Japan, similarly as the method in Example 5-1 disclosed in International Publication No. Wo 2004/042056 to prepare an expression vector for membrane-associated polypeptide mAgK114-1, named pCD/mAgK114, and an expression vector for secretory polypeptide mAgK114-1b, named pCD/mAgK114b.

### EXAMPLE 6-2

### Transformation of COS-1 Cells and Expression of Mouse Membrane-Associated Polypeptide mAqK114-1

COS-1 cells were transformed similarly as indicated below by introducing the expression vector pCD/mAgK114 or the pCD/mAgK114b obtained in Example 6-1 using “LIPOFECTAMINE 2000”, an agent for gene introduction used for lipofection commercialized by Invitrogen Japan K.K., Tokyo, Japan. A 50 pl solution was prepared by diluting 0.8 μg of a plasmid DNA with Opti-MEM, a medium commercialized by Invitrogen Japan K.K., Tokyo, Japan, and a 50 μl solution was prepared by diluting 3 μg of LIPOFECTAMINE with Opti-MEM. These solutions were allowed to stand at ambient temperature for five minutes and then mixed to react them for 20 min to form a DNA-LIPOFECTAMINE complex. COS-1 cells were inoculated to a 24-well plate with D-MEM supplemented with 10% (v/v) fetal calf serum to give a cell density of 6×10⁴ cells/well and cultured overnight, and then the culture supernatant in each well was removed and replaced with 100 μl of the DNA-LIPOFECTAMINE and 0.4 ml of a serum-free D-MEM. As a control, only an expression vector pCDM8 was transfected. These transfected cells were cultured at 37° C. for five hours under 5% (v/v) CO₂ conditions to obtain COS-1 cells constructed to express membrane-associated polypeptide mAgK114-1 or secretory polypeptide mAgK114-1b.

### EXAMPLE 6-3

### Action of Enhancing the Production of IL-6 in Mouse Embryonic Fibroblasts (MEF Cells) and Macrophage Cells when Co-Cultured with COS-1 Cells Constructed to Express Membrane-Associated Polypeptide mAqK114-1 or Secretory Polypeptide mAqK114-1b

From the cell cultures of COS-1 cells obtained in Example 6-2, which had expressed membrane-associated polypeptide mAgK114-1 or secretory polypeptide mAgK114-1b, were removed supernatants which were then replaced with two milliliters of D-MEM supplemented with 10% (v/v) fetal calf serum, which had been prepared to contain 1×10⁵ cells/well of MEF cells and 4×10⁵ cells/well of J774A.1 cells, followed by simultaneously initiating co-culture. On three days after the co-culture, the supernatant in each well of the culture plate was collected and assayed for IL-6 concentration by EIA. As a control, COS-1 cells with no expression of any of the above polypeptides were treated similarly as above. The result is in Table 10.

TABLE 10

| | |
|---|---|
| | Production level |
| Concentration of | (pg/ml) of IL-6* |

| polypeptide (μg/ml) | MEF cells | J774A.1 cells |
|---|---|---|
| Control** | ND*** | ND*** |
| mAgK114-1 | 3541 | 1179 |
| (membrane-associated type) | | |
| mAgK114-1b | 1818 | 810 |
| (secretory type) | | |

*Interleukin 6
**COS-1 cells alone
***Below detection limit of 25.0 pg/ml

As evident from the result in Table 10, it was revealed that any of the COS-1 cells, which had expressed membrane-associated polypeptide mAgK114-1 or secretory polypeptide mAgK114-1b, enhance the production of IL-6 in both MEF cells and J774A.1 cells when co-cultured therewith. The production level of IL-6 in MEF cells, which had expressed membrane-associated polypeptide mAgK114-1, and that of IL-6 in MEF cells, which had expressed secretory polypeptide mAgK114-1b, were respectively increased by about 140-folds and about 70-folds higher than those of their respective controls. While in the case of the production level of IL-6 in J774A.1 cells, which had expressed membrane-associated polypeptide mAgK114-1, and that of IL-6 in J774A.1 cells, which had expressed secretory polypeptide mAgK114-1b, were respectively increased by about 50-folds and about 30-folds higher than those of their respective controls. It was confirmed that mouse membrane-associated polypeptide mAgK114-1, which has the amino acid sequence of SEQ ID NO:3, has an action of enhancing the production of IL-6.

### EXAMPLE 7

### Action of Enhancing the Production of Cytokines in Wounded Sites of Mouse Skin by Mouse Polypeptide mAqK114-b

Sixteen female ICR/CD-1 mice, six weeks of age, were anesthetized, depilated their dorsum skin, and developed a linear wound with a length of 8 mm on the midline of each mouse. Among these mice, eight mice were administered with a polypeptide having the amino acid sequence of SEQ ID NO:2, i.e., a mouse polypeptide mAgK114-1b, which had been prepared and purified by the method in Example 10 disclosed in International Publication No. WO 2004/042056, to their wounded sites at four times in total just after having been wounded and at 6, 24 and 30 hours after the wounding and at a dose of five micrograms of the polypeptide in 10 μl of phosphate buffer per wounded site. As a control, the remaining eight mice were respectively administered with only phosphate buffer similarly as above. On days 1 and 4 after developing the wound, about 500 mg of the skin tissue was excised from each of the wounded sites of four mice in each group, suspended in one milliliter of 20 mM Tris-HCl buffer containing 150 mM salt, 1% of “NONIDET P-40”, a product name of surfactant commercialized by Nacalai Tesque Inc., Tokyo, Japan, 10 mM EDTA, 10% glycerol, and “COMPLETE”, a product name of protease inhibitor commercialized by Roche Diagnostics K.K., Tokyo, Japan, repeatedly subjected to freezing and thawing thrice, and treated with a homogenizer to prepare a homogenate. Assaying each supernatant obtained by centrifuging the homogenate for the amount of cytokines, i.e., transforming growth factor-β (TGF-β) and vascular endothelial growth factor (VEGF), with an EIA kit commercialized by R & D Systems, Minneapolis, Minn., USA, the assayed data were respectively converted into a protein amount per one milligram of each supernatant. The result is in Table 11.

TABLE 11

| | |
|---|---|
| | Production level* of |
| Dose of | cytokine |

| Elapsed days | polypeptide | TGF-β** | VEGF*** |
|---|---|---|---|
| (days) | (μg, frequency) | (ng/mg) | (pg/mg) |
| 1 | — | 53.2 | 12.4 |
| | 5 μg, 2 shots | 172.8 | 70.4 |
| 4 | — | 182.6 | 96.5 |
| | 5 μg, 4 shots | 206.4 | 167.5 |

*Production level per mg protein in the supernatant of homogenate (mean value of four samples)
**Transforming growth factor-β (TGF-β)
***Vascular endothelial growth factor (VEGF)

As evident from the result in Table 11, the group of mice, administered with the mouse polypeptide mAgK114-1b, remarkably increased the production levels of TGF-β and VEGF by about 3-folds and about 5-folds, respectively, on day one after the polypeptide administration; and remarkably increased the production level of VEGF by about 2-folds on day four after the polypeptide administration. The polypeptide used in the present invention enhances the production of cytokines such as TGF-β and VEGF, and it is useful in the field of pharmaceuticals, for example, as an agent for repairing tissues used in treating wounds.

### EXAMPLE 8

### Inhibitory Action of Cell Infiltration in Mouse Model Suffering from Atopic Dermatitis by Mouse Polypeptide mAgK114-1b

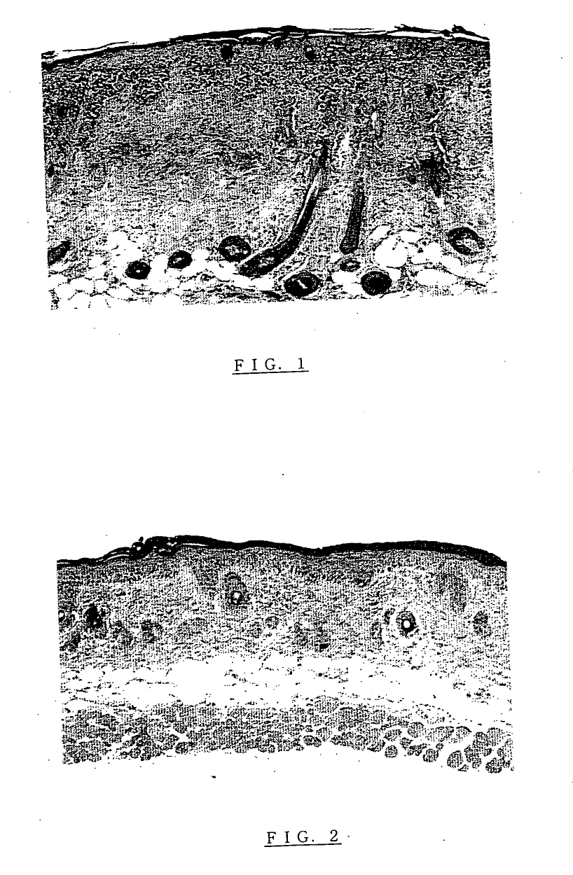

According to conventional manner, a mouse model suffering from atopic dermatitis induced by applying picryl chloride: The mouse model was prepared by applying 150 μl of a solution containing 5% picryl chloride in ethanol/acetone (=4:1 by volume) to a mouse at its abdominal cutaneous epithelium and then, from five days after the application, applying 150 μl of a solution containing 1% picryl chloride in olive oil to the dorsum site of the mouse eight times at regular intervals of one week. A polypeptide having the amino acid sequence of SEQ ID NO:2, prepared and purified by the method disclosed in Example 10 in International Publication No. WO 2004/042056, i.e., mouse polypeptide mAgK114-1b was prepared into an ointment in a conventional manner, which was then administered to the dorsum cutaneous epithelium of the mouse at a dose of 2.5 mg/kg body weight of mouse at a frequency of three times per week (17 shots in total) during 17 to 55 days after the application of the solution containing 5% picryl chloride in ethanol/acetone. On day two after the final administration of mAgK114-1b, a skin slice was prepared from the mouse and evaluated based on macroscopic observation. As a control, there was provided a skin slice from a mouse suffering from atopic dermatitis, which had been applied with an ointment free of mAgK114-1b. As a result, intraepidermally infiltrated cells (black dots in the upper part of the cross sectional view) were observed in the skin slice of the control mouse (FIG. 1), while no cells intraepidermally infiltrated as such were observed in the skin slice of the mouse administered with mAgK114-1b (FIG. 2). These results revealed that the administration of mAgK114-1b to the skin inhibits cell infiltration into the skin and exerts an action of alleviating dermatitis.

### EXAMPLE 9

### Action of Inhibiting the Production of IgE in Mouse Model Suffering from Atopic Dermatitis by Mouse Polypeptide mAqK114-1b

mAgK114-1b was administered to BALB/c mice, eight weeks of age, grouped into five heads per group, at a dose of 1 μg or 10 μg per head at a frequency of three shots per week. As a control, mouse albumin was administered to mice at a dose of 10 μg per head similarly as above. After the third administration in the first administration week, the mice were applied with 150 μl of a solution containing 5% picryl chloride in ethanol/acetone (=4:1 by volume) at their dorsum parts and further applied, as re-sensitization, to their auriculae with a solution containing 1% picryl chloride in ethanol/acetone (=4:1 by volume) on days 5 (first re-sensitization), 12 (second re-sensitization), 19 (third re-sensitization), and 26 (fourth re-sensitization) after the first administration. On day six every after the second, third and fourth re-sensitizations, mice were collected blood in a small amount from their tail veins, followed by assaying the collected blood for IgE level by a conventional EIA method. The result is in Table 12.

TABLE 12

| | IgE Level (μg/ml) | | |
|---|---|---|---|
| | Re-sensitization | | |
| Sample | Second | Third | Fourth |
| Mouse serum albumin | 0.37 | 2.2 | 16 |
| 10 μg/ml (Control) | | | |
| mAgK114-1b | 0.68 | 2.9 | 11 |
| 1 μg | | | |
| mAgK114-1b | 0.61 | 3.2 | 8.9 |
| 10 μg | | | |

As evident from the result in Table 12, the IgE level in the blood of mice was remarkably increased from the level of 2.2 μg/ml to 16 μg/ml when evaluated after the fourth re-sensitization of picryl chloride, while mouse polypeptide mAgK114-1b inhibited the increment of IgE level down to 8.9 or 11 μg/ml. The result indicates that mouse polypeptide mAgK114-1b has an inhibitory effect on the increment of IgE production induced remarkably when in atopic dermatitis, and thus the polypeptide is useful as an agent for alleviating allergy.

### EXAMPLE 10

### Liquid

The purified human polypeptide hAgK114-1aFL used in Example 2 and human serum albumin were dissolved in physiological saline to give respective concentrations of 0.1% by weight, followed by sterilizing the solution with a membrane filter to obtain a liquid. The product is useful in the field of pharmaceuticals as an agent for increasing platelet blood count and an inhibitory agent for allergy.

### INDUSTRIAL APPLICABILITY

As described above, the agent for enhancing the production of cytokines and/or chemokines according to the present invention enhances the production of IL-6, M-CSF and IL-1 and it is useful as an agent for increasing the blood cells and platelet in the field of pharmaceuticals to be used to increase the blood cells decreased by bone marrow transplantation, chemotherapy or radiotherapy in treating cancers, etc; or proliferating hemopoietic cells ex vivo. Since the agent enhances the production of cytokines such as TNF-α, TGF-62 and VEGF, it is useful as an agent for repairing wounded tissues. Further, the agent for enhancing the production of cytokines and/or chemokines according to the present invention also enhances the production of chemokines such as SDF-1, RANTES, IL-8 and MIP and it is useful as an agent for alleviating allergy such as atopic dermatitis and a phylactic agent for microorganisms.

1-9. (canceled)

10. An agent for enhancing the production of cytokines and/or chemokines, which comprises as an effective ingredient a polypeptide having any one of the amino acid sequences of SEQ ID Nos:1 to 3, where one or more amino acids are in any one of the SEQ ID Nos:1 to 3 are deleted or replaced with other amino acid(s) and/or one or more amino acids are added to any one of the amino acid sequences of SEQ ID NOs:1 to 3, without substantially losing the biological activity of the polypeptide.

11. The agent of claim 10, wherein said polypeptide enhances the production of cytokines and/or chemokines in hematopoietic cells comprising mammalian mesenchyme cells, fibroblasts, epithelial cells, or macrophagecells.

12. The agent of claim 10, wherein said polypeptide is obtainable by a step of artificially expressing a DNA having any one of the nucleotide sequences of SEQ ID NOs:4 to 6, any one of the nucleotide sequences of SEQ ID NOs:4 to 6 where one or more bases thereof are replaced with other bases, without substantially altering the amino acid sequences encoded by said DNA, or a complementary nucleotide sequence thereunto.

13. The agent of claim 10, which is one or more members selected from the group consisting of interleukin-6 (IL-6), macrophage colony-stimulating factor (M-CSF), interleukin-1 (IL-1), tumor necrosis factor-α (TNF-α), transforming growth factor-β (TGF-β), and vascular endothelial growth factor (VEGF).

14. The agent of claim 10, which is one or more members selected from the group consisting of stromal cell derived factor-1 (SDF-1); interleukin-8 (IL-8); regulated on activation, normal T-cell expressed and secreted (RANTES); and macrophage inflammatory protein (MIP).

15. A pharmaceutical composition, which comprises, as an effective ingredient, the production enhancer for cytokines and/or chemokines of claim 10 and a pharmaceutically acceptable carrier.

16. An agent for increasing platelet blood count, which comprises, as an effective ingredient, the production enhancer for cytokines and/or chemokines of claim 10.

17. An allergy inhibitory agent, which comprises, as an effective ingredient, a polypeptide having an amino acid sequence of any one of the amino acid sequences of SEQ ID NOs:1 to 3.
